(19) United States
(12) Patent Application Publication
(10) Pub. No.: US 2005/0282786 A1
(43) Pub. Date: Dec. 22, 2005

(54) Method for reducing c-rective protein levels with non-antibacterial tetracycline formulations

(76) Inventors: David Brown, Bronxville, NY (US); Lorne M. Golub, Smithtown, NY (US); Hsi-Ming Lee, Setauket, NY (US); Robert Greenwald, Melville, NY (US)

Correspondence Address: Ronald Baron;Hoffmann & Baron, 6900 Jericho Turnpike, Syosset, NY, 11791, US

(21) Appl. No.: 10/519,534

(22) PCT Filed: Jul. 11, 2003
(86) PCT No.: PCT/US03/21740

§ 371 Date: Aug. 4, 2005

(30) Foreign Application Priority Data
Jul. 12, 2002 (US) 60395466

(51) Int. Cl. A61K 31/65

(52) U.S. Cl. 514/152

(57) ABSTRACT

The present invention is for a method for decreasing C-reactive protein levels (CRP) in a mammal in need thereof. The method comprises administering an effective amount of a non-antibacterial tetracycline formulation, to the mammal. In one embodiment, the non-antibacterial tetracycline formulation is a non-antibacterial amount of an antibacterial tetracycline. In another embodiment, the non-antibacterial tetracycline formulation is a non-antibacterial tetracycline.

### BACKGROUND OF THE INVENTION

C-reactive protein (CRP) is a special type of protein referred to as an acute phase reactant. Acute phase reactants, such as CRP, are released by the body in response to acute injury, infection or other inflammatory conditions, such as, for example, atherosclerosis.

Atherosclerosis is a condition in which atheromatous plaques form in the arteries. Atheromatous plaques are deposits, or degenerative accumulations, of lipids on the innermost layer of the wall of an artery. Such plaques contain inflammatory cells. The rupture of atheromatous plaques is thought to be the mechanism for acute myocardial infarction (e.g. heart attack).

The release of acute phase reactants, such as CRP, in response to inflammation, has been proposed as a potential marker of coronary artery diseases, due to, for example, atherosclerosis. Accordingly, current research is focusing on developing drugs that inhibit CRP, and thus, decrease the incidence of such diseases. See, Taubes, Gary, Does Inflammation Cut to the Heart of the Matter?, Science, 12 April 2002; 296: 242-245. For example, recent studies have shown that treatment with pravastatin, an HMG-CoA reductase inhibitor (i.e. statin), appears to result in reduced levels of CRP. Ridker, P., Nader, R, et al. Long Term Effects of Pravastin on Plasma Concentration of C-Reactive Protein, Circulation, 1999;100:230-235.

However, HMG-CoA reductase inhibitors, such as pravastatin, are associated with numerous side effects. These side effects include constipation, stomach pain, nausea and vomiting.

Therefore, the prior art treatments for reducing CRP levels are limited and not without adverse effects. There is a need for novel, alternate, and superior treatments for reducing CRP levels.

The compound tetracycline is a member of a class of antibiotic compounds that is referred to as the tetracyclines, tetracycline compounds, tetracycline derivatives and the like. The compound tetracycline exhibits the following general structure:

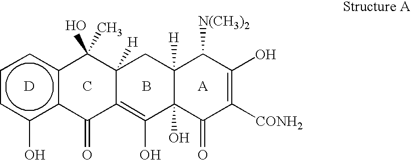

The numbering system of the tetracycline ring nucleus is as follows:

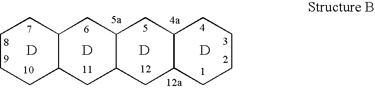

Tetracycline, as well as the terramycin and aureomycin derivatives, exist in nature, and are well known antibiotics. Natural tetracyclines may be modified without losing their antibiotic properties, although certain elements must be retained. The modifications that may and may not be made to the basic tetracycline structure have been reviewed by Mitscher in The Chemistry of Tetracyclines, Chapter 6, Marcel Dekker, Publishers, New York (1978). According to Mitscher, the substituents at positions 5-9 of the tetracycline ring system may be modified without the complete loss of antibiotic properties.

Changes to the basic ring system or replacement of the substituents at positions 4 and 10-12a, however, generally lead to synthetic tetracyclines with substantially less or effectively no antimicrobial activity. Some examples of chemically modified non-antibacterial tetracyclines (hereinafter CMTs) are 4-dedimethylaminotetracyline, 4-dedimethylaminosancycline (6-demethyl-6-deoxy-4-dedimethylaminotetracycline), 4-dedimethylaminominocycline (7-dimethylamino6-demethyl-6-deoxy-4-dedimethylaminotetracycline), and 4-dedimethylaminodoxycycline (5-hydroxy-6-deoxy-dedimethylaminotetracycline).

In addition to their antimicrobial properties, tetracyclines have been described as having a number of other uses. For example, tetracyclines are also known to inhibit the activity of collagen destructive enzymes produced by mammalian (including human) cells and tissues by non-antibiotic mechanisms. Such enzymes include the matrix metalloproteinases (MMPs), including collagenases (MMP-1, MMP-8 and MMP-13), gelatinases (MMP-2 and MMP-9), and others (e.g. MMP-12, MMP-14). See Golub et al., J. Periodont. Res. 20:12-23 (1985); Golub et al. Crit. Revs. Oral Biol. Med. 2:297-322 (1991); U.S. Pat. Nos. 4,666,897; 4,704,383; 4,935,411; 4,9354,412. Also, tetracyclines have been known to inhibit wasting and protein degradation in mammalian skeletal muscle, U.S. Pat. No. 5,045,538, to inhibit inducible NO synthase, U.S. Pat. Nos. 6,043,231 and 5,523,297, and phospholipase A₂, U.S. Pat. Nos. 5,789,395 and 5,919,775, and to enhance IL-10 production in mammalian cells. These properties cause the tetracyclines to be useful in treating a number of diseases.

The object of this invention is to provide a method for reducing C-reactive protein levels in a mammal in need thereof.

### SUMMARY OF THE INVENTION

It has now been discovered that this and other objectives can be achieved by the present invention. The present invention provides a method for decreasing C-reactive protein levels (CRP) in a mammal in need thereof. The method comprises admninistering an effective amount of a non-antibacterial tetracycline formulation, to the mammal.

In one embodiment, the non-antibacterial tetracycline formulation is a non-antibacterial amount of an antibacterial tetracycline. In another embodiment, the non-antibacterial tetracycline formulation is a non-antibacterial tetracycline.

### DETAILED DESCRIPTION

The invention relates to decreasing C-reactive protein levels by administering a non-antibacterial tetracycline formulation.

In one embodiment of the invention, the non-antibacterial tetracycline formulation is an antibacterial tetracycline compound administered in a non-antibacterial amount, as will be discussed below. For this embodiment, the tetracycline may be any such tetracycline having clinically significant antibacterial activity.

Some examples of antibacterial tetracyclines include tetracycline, as well as the 5-OH (oxytetracycline, e.g. Terramycin) and 7-Cl (chlorotetracycline, e.g. Aureomycin) derivatives, which exist in nature. Semi-synthetic tetracyclines, which include, for example, doxycycline, minocycline and sancycline, can also be used for this embodiment. Examples also include demeclocycline and lymecycline.

In another embodiment of the invention, the non-antibacterial tetracycline formulation is a non-antibacterial tetracycline compound. Non-antibiotic tetracycline compounds are structurally related to the antibiotic tetracyclines, but have had their antibiotic activity substantially or completely eliminated by chemical modification, as mentioned above. For example, non-antibiotic tetracycline compounds are incapable of achieving antibiotic activity comparable to that of doxycycline unless the concentration of the non-antibiotic tetracycline is at least about ten times, preferably at least about twenty five times, greater than that of doxycycline.

One such group of chemically modified non-antibacterial tetracyclines (CMT's) includes any of the 4-dedimethylaminotetracycline derivatives, for example, 4-dedimethylaminosancycline (CMT-3), 4-dedimethylaminodoxycycline (CMT-8) and 4-dedimethylaminominocycline (CMT-10).

Some additional examples of generic and specific chemically modified, non-antibiotic tetracycline compounds that are suitable for use in the method of the invention are found in PCT/US01/16272. All such generic and specific compounds are incorporated herein by reference.

Some preferred examples of suitable 4-dedimethylaminotetracycline derivatives include the following general formulae (1) through (IV):

### General Formula (I)

Structure A represents the 4-dedimethylaminosancycline (CMT-3) derivatives

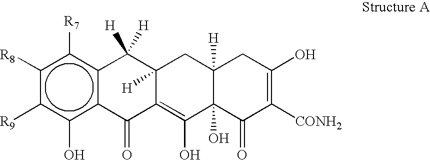

wherein R7, R8, and R9 taken together in each case, have the following meanings:

| | R7 | R8 | R9 |
|---|---|---|---|
| | azido | hydrogen | hydrogen |
| | dimethylamino | hydrogen | azido |
| | hydrogen | hydrogen | azido |
| | dimethylamino | hydrogen | amino |
| | acylamino | hydrogen | hydrogen |
| | amino | hydrogen | nitro |
| | hydrogen | hydrogen | (N,Ndimethyl)glycylamino |
| | amino | hydrogen | amino |
| | hydrogen | hydrogen | ethoxythiocarbonylthio |
| | dimethylamino | hydrogen | acylamino |
| | dimethylamino | hydrogen | diazonium |
| | dimethylamino | chloro | amino |
| | hydrogen | chloro | amino |
| | amino | chloro | amino |
| | acylamino | chloro | acylamino |
| | amino | chloro | hydrogen |
| | acylamino | chloro | hydrogen |
| | monoalkylamino | chloro | amino |
| | nitro | chloro | amino |
| | dimethylamino | chloro | acylamino |
| | dimethylamino | chloro | dimethylamino |
| | acylamino | hydrogen | hydrogen |
| | hydrogen | hydrogen | acylamino |
| (CMT-301) | bromo | hydrogen | hydrogen |
| (CMT-302) | nitro | hydrogen | hydrogen |
| (CMT-303) | hydrogen | hydrogen | nitro |
| (CMT-304) | acetamido | hydrogen | hydrogen |
| (CMT-305) | hydrogen | hydrogen | acetamido |
| (CMT-306) | hydrogen | hydrogen | dimethylamino |
| (CMT-307) | amino | hydrogen | hydrogen |
| (CMT-308) | hydrogen | hydrogen | amino |
| (CMT-309) | hydrogen | hydrogen | dimethylaminoacetamido |
| (CMT-310) | dimethylamino | hydrogen | hydrogen |
| (CMT-311) | hydrogen | hydrogen | palmitamide |

| | R7 | R8 | R9 | R2 |
|---|---|---|---|---|
| (CMT- | hydrogen | hydrogen | hydrogen | CONHCH₂-pyrrolidin-1-yl |
| 312) | | | | |
| (CMT- | hydrogen | hydrogen | hydrogen | CONHCH₂-piperadin-1-yl |
| 313) | | | | |
| (CMT- | hydrogen | hydrogen | hydrogen | CONHCH₂-morpholin-1-yl |
| 314) | | | | |
| (CMT- | hydrogen | hydrogen | hydrogen | CONHCH₂-piperazin-1-yl |
| 315) | | | | |

### General Formula (II)

Structures B through E represent the 4-dedimethylaminodoxycycline (CMT-8) derivatives

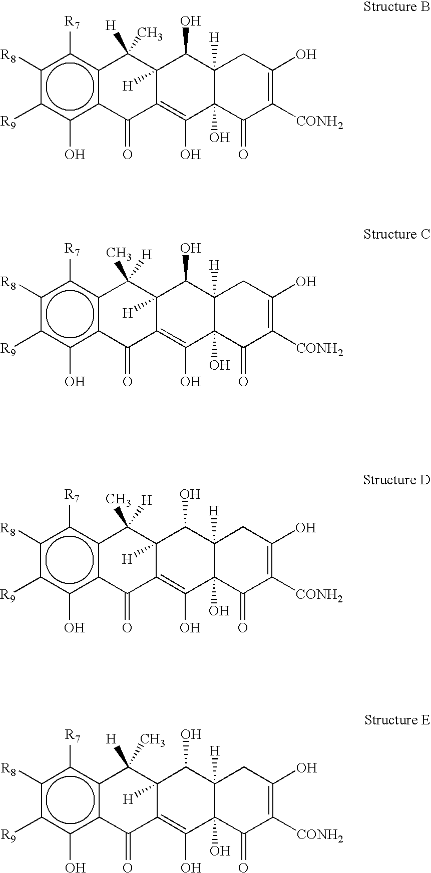

wherein R7, R8, and R9 taken together in each case, have the following meanings:

| | R7 | | R8 | R9 |
|---|---|---|---|---|
| | azido | | hydrogen | hydrogen |
| | dimethylamino | | hydrogen | azido |
| | hydrogen | | hydrogen | azido |
| | dimethylamino | | hydrogen | amino |
| | acylamino | | hydrogen | hydrogen |
| | hydrogen | | hydrogen | acylamino |
| | amino | | hydrogen | nitro |
| | hydrogen | | hydrogen | (N,N-dimethyl)glycylamino |
| | amino | | hydrogen | amino |
| | hydrogen | | hydrogen | ethoxythiocarbonylthio |
| | dimethylamino | | hydrogen | acylamino |
| | hydrogen | | hydrogen | diazonium |
| | diazonium | | hydrogen | hydrogen |
| | ethoxythiocarbonylthio | | hydrogen | hydrogen |
| | dimethylamino | | chloro | amino |
| | amino | | chloro | amino |
| | acylamino | | chloro | acylamino |
| | hydrogen | | chloro | amino |
| | amino | | chloro | hydrogen |
| | acylamino | | chloro | hydrogen |
| | monoalkylamino | | chloro | amino |
| | nitro | | chloro | amino |
| (CMT- | hydrogen | | hydrogen | acetamido |
| 801) | | | | |
| (CMT- | hydrogen | | hydrogen | dimethylaminoacetamido |
| 802) | | | | |
| (CMT- | hydrogen | | hydrogen | palmitamide |
| 803) | | | | |
| (CMT- | hydrogen | | hydrogen | nitro |
| 804) | | | | |
| (CMT- | hydrogen | | hydrogen | amino |
| 805) | | | | |
| (CMT- | hydrogen | | hydrogen | dimethylamino |
| 806) | | | | |
| | R7 | R8 | R9 | R2 |
| (CMT- | hydrogen | hydrogen | hydrogen | CONHCH₂-pyrrolidin-1-yl |
| 807) | | | | |
| (CMT- | hydrogen | hydrogen | hydrogen | CONHCH₂-piperadin-1-yl |
| 808) | | | | |
| (CMT- | hydrogen | hydrogen | hydrogen | CONHCH₂-piperazine-1-yl |
| 809) | | | | |

### General Formula (III)

Structure F represents the 4-dedimethylaminominocycline (CMT-10) derivatives 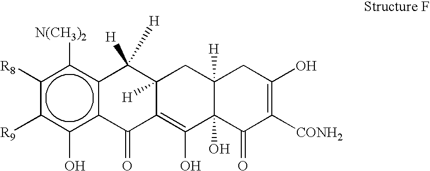
wherein R8 is hydrogen or halogen and R9 is selected from the group consisting of nitro (CMT-1002), (N,N-dimethyl)glycylamino, ethoxythiocarbonylthio. A compound related to structure F has a 7-trimethylammonium group instead of the 7-diemthylamino group, i.e. 7-trimethylammoniumsancycline (CMT-1001), and

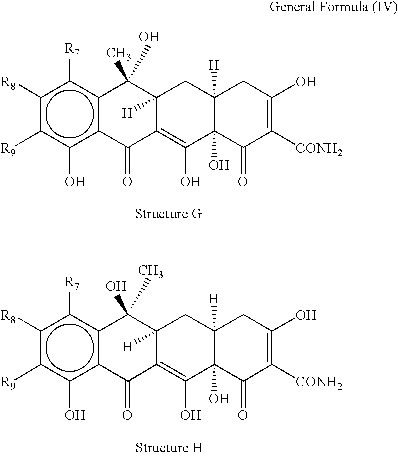

wherein R7, R8, and R9 taken together in each case, have the following meanings:

| R7 | R8 | R9 |
|---|---|---|
| amino | hydrogen | hydrogen |
| nitro | hydrogen | hydrogen |
| azido | hydrogen | hydrogen |
| dimethylamino | hydrogen | azido |
| hydrogen | hydrogen | amino |
| hydrogen | hydrogen | azido |
| hydrogen | hydrogen | nitro |
| bromo | hydrogen | hydrogen |
| dimethylamino | hydrogen | amino |
| acylamino | hydrogen | hydrogen |
| hydrogen | hydrogen | acylamino |
| amino | hydrogen | nitro |
| hydrogen | hydrogen | (N,N-dimethyl)glycylamino |
| amino | hydrogen | amino |
| diethylamino | hydrogen | hydrogen |
| hydrogen | hydrogen | ethoxythiocarbonylthio |
| dimethylamino | hydrogen | methylamino |
| dimethylamino | hydrogen | acylamino |
| dimethylamino | chloro | amino |
| amino | chloro | amino |
| acylamino | chloro | acylamino |
| hydrogen | chloro | amino |
| amino | chloro | hydrogen |
| acylamino | chloro | hydrogen |
| monoalkylamino | chloro | amino |
| nitro | chloro | amino |

Additional CMT's for purposes of the invention include, 4-dedimethylaminotetracycline (CMT-1), tetracycline nitrile (CMT-2), 4-dedimethylaminochlorotetracycline (CMT-4), 4-dedimethylamino-4-hydroxytetracycline (CMT), 2a-dehydroxy-4-dedimethylaminotetracycline (CMT-7), and 1-deoxy-12a-dehydroxy-4-dedimethylaminotetracycline (CMT-9).

The chemically modified tetracyclines can be made by methods known in the art. See, for example, Mitscher, L. A., The Chemistry of the Tetracycline Antibiotics, Marcel Dekker, New York (1978), Ch. 6, and U.S. Pat. Nos. 4,704,383 and 5,532,227.

The invention also includes pharmaceutically acceptable salts of the above disclosed compounds. The present invention embraces salts, including acid-addition and metal salts of the 4-dedimethylaminotetracycline compounds described herein. Such salts are formed by well known procedures. By “pharmaceutically acceptable salts” is meant salts that do not substantially contribute to the toxicity of the compound.

Some examples of suitable salts include salts of basic tetracycline compounds and mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g. p-toluenesulfonic acids, and the like.

After preparation, the novel compounds of the present invention can be conveniently purified by standard methods known in the art. Some suitable examples include crystallization from a suitable solvent or partition-column chromatography.

The preferred pharmaceutical composition for use in the method of the invention includes a combination of the tetracycline compound in a suitable pharmaceutical carrier (vehicle) or excipient as understood by practitioners in the art. Examples of carriers and excipients include starch, milk, sugar, certain types of clay, gelatin, stearic acid or salts thereof magnesium or calcium stearate, talc, vegetable fats or oils, gums and glycols.

The tetracycline compounds of the invention may be administered by methods known in the art, typically, systemically. Systemic administration can be enteral or parenteral. Enteral administration is a preferred route of delivery of the tetracycline, and compositions including the tetracycline compound with appropriate diluents, carriers, and the like are readily formulated. Liquid or solid (e.g., tablets, gelatin capsules) formulations can be employed.

Administration can also be accomplished by a nebulizer or liquid mist. Nebulization is a preferred route of delivery of the tetracycline in situations where the respiratory system is particularly infected. By utilizing a nebulizer, the tetracycline is taken directly into the individuals respiratory system through inspiration.

Parenteral administration of the tetracycline compounds of the invention (e.g., intravenous, intramuscular, subcutaneous injection) is also contemplated. Formulations using conventional diluents, carriers, etc. such as are known in the art can be employed to deliver the compound.

The tetracycline compound may be administered to mammals by sustained release, as is known in the art. Sustained release administration is a method of drug delivery to achieve a certain level of the drug over a particular period of time.

The amount of tetracycline compound administered is any amount effective for decreasing CRP levels in the mammal in need thereof. The actual preferred amounts of tetracycline compound in a specified case will vary according to the particular compositions formulated, the mode of application, and the particular subject being treated. The appropriate dose of the tetracycline compound can readily be determined by those skilled in the art.

The minimal amount of the tetracycline compound administered to a human is the lowest amount capable of providing effective treatment of the conditions. Effective treatment is a decrease in CRP levels, of the mammal.

The maximal amount for a mammal is the highest amount that does not cause undesirable or intolerable side effects. Such doses can be readily determined by those skilled in the art.

The amount of an antibacterial tetracycline is an amount that has substantially no antibacterial activity, i.e. an amount that does not significantly prevent the growth of bacteria For example, tetracycline compounds that have significant antibacterial activity may be administered in an amount which is 10-80% of the antibacterial amount. More preferably, the antibacterial tetracycline compound is administered in an amount which is 40-70% of the antibacterial amount.

The amount of tetracycline administered may be measured, for example, by a daily dose or by serum level. Some examples of non-antibiotic daily doses of antibiotic tetracyclines, based on steady-state pharmacokinetics, are as follows: 20 mg/twice a day for doxycycline; 38 mg of minocycline one, two, three or four times a day, 60 mg of tetracycline one, two, three or four times a day, 1000 mg/day of oxytetracycline, 600 mg/day of demeclocycline and 600 mg/day of lymecycline.

In a preferred embodiment, doxycycline is administered in a daily amount of from about 10 to about 60 milligrams, preferably 30 to 60 milligrams, but maintains a concentration in human plasma below the threshold for a significant antibiotic effect.

In an especially preferred embodiment, doxycycline hyclate is administered at a 20 milligram dose twice daily. Such a formulation is sold for the treatment of periodontal disease by CollaGenex Pharmaceuticals, Inc. of Newtown, Pa. under the trademark Periostat®.

Antibiotic serum levels are also known in the art. For example, a single dose of two 100 mg minocycline HCl tablets administered to an adult human results in minocycline serum levels ranging from 0.74 to 4.45 μg/ml over a period of an hour. The average level is 2.24 μg/ml.

Two hundred and fifty milligrams of tetracycline HCl administered every six hours over a twenty-four hour period produces a peak plasma concentration of approximately 3 μg/ml. Five hundred milligrams of tetracycline HCl administered every six hours over a twenty-four hour period produces a serum concentration level of 4 to 5 μg/ml.

In general, the tetracycline compound is administered in an amount which results in a serum concentration between about 0.1 and 10.0 μg/ml, more preferably between 0.3 and 5.0 μg/ml. For example, doxycycline, in a non-antibacterial formulation, is administered in an amount which results in a serum concentration between about 0.1 and 0.8 μg/ml, more preferably between 0.4 and 0.7 μg/ml.

Non-antibacterial tetracycline compounds can be used in higher amounts than antibacterial tetracyclines, while avoiding the indiscriminate killing of bacteria, and the emergence of resistant bacteria. For example, 6-demethyl-6-deoxy-4-dedimethylaminotetracycline (CMT-3) may be administered in doses of about 10 to about 200 mg/day, or in amounts that result in serum levels in humans of about 1.0 μg/ml to about 10 μg/ml. For example, a dose of about 10 to about 20 mg/day produces serum levels in humans of about 1.0 μg/ml.

For example, CMTs can be systemically administered in a mammal in a minimal amount of about 0.05 mg/kg/day to about 0.3 mg/kg/day, and a maximal amount of about 18 mg/kg/day to about 60 mg/kg/day. The practitioner is guided by skill and knowledge in the field, and the present invention includes, without limitation, dosages that are effective to achieve the desired antibacterial activity.

The tetracyclines of the present invention decrease CRP levels in mammals in need thereof. CRP, as discussed above, is a special type of protein produced during inflammation.

A mammal in need of decreasing CRP levels is any mammal that has an elevated CRP level. For example, a mammal having a condition associated with inflammation will have an elevated CRP level. Conditions associated with inflammation include, for example, cardiac conditions, cerebrovascular disease, arthritis, asthma, periodontitis, cancer, and lupus.

Cardiovascular conditions include, for example, myocardial infarction, atherosclerosis, and angina Cerebrovascular disease includes stroke and aneurysm.

A mammal which can benefit from the methods of the present invention could be any mammal. Categories of mammals include, for example, humans, farm animals, domestic animals, laboratory animals, etc. Some examples of farm animals include cows, pigs, horses, goats, etc. Some examples of domestic animals include dogs, cats, etc. Some examples of laboratory animals include rats, mice, rabbits, guinea pigs, etc.

### EXAMPLES

The following exemplary data serves to provide further appreciation of the invention but are not meant in any way to restrict the effective scope of the invention.

A prospective, randomized study was conducted over six (6) months to investigate the effectiveness of low-dose doxycycline (LDD) versus placebo, in the prevention of subsequent plaque rupture events in patients enrolled after an initial acute coronary syndrome.

Biochemical markers of inflammation were assessed at study entry and after six (6) months of therapy in a subset of patients. A total of thirty (30) patients completed the study of whom thirteen (13) were randomized to placebo and seventeen (17) to LDD. There were no significant differences in age, male gender, hypertension, diabetes, smoking, previous cardiac history, extent of coronary disease, presentation with acute myocardial infarction or unstable angina, or percutaneous coronary intervention between LDD and placebo treated patients.

At six months clinical follow-up, there was no difference in the composite endpoint of cardiovascular death, myocardial infarction or troponin-positive unstable angina in LDD compared to placebo treated patients. As demonstrated in Table I, C-reactive protein (CRP) levels were reduced by 46% from 4.8 μg/ml to 2.6 μg/ml (P<0.05) among patients randomized to LDD. In placebo-treated patients, CRP was 5.2 μg/ml at study entry and 4.9 μg/ml at six months (P=not significant (n.s.)).

TABLE I
The Effect of Low-Dose Doxycycline (LDD) on C-Reactive Protein in
Patients With Acute Myocardial Syndrome: Preliminary Data¹

| | | | Low-Dose | |
|---|---|---|---|---|
| | Placebo | | Doxycycline | |
| | (n = 13 subjects) | | (n = 17 subjects) | |
| Baseline CRP level | 5.2 ± 0.8 | μg/ml | 4.8 ± 0.6 | μg/ml |
| 6-Month CRP level | 4.9 ± 0.7² | μg/ml | 2.6 ± 0.4³ | μg/ml |

| | | |
|---|---|---|
| Reduction Due | 5% | 45% |
| To Treatment | | |

¹Each value represents the mean ± S.E.M.
²not significant (n.s.) comparing six-month values to baseline values.
³P < 0.05

Table II demonstrates the preferential efficacy of LDD at decreasing CRP levels in patients having higher baseline CRP levels. LDD-treated patients having lower baseline CRP levels showed a 23% reduction (3.0 μg/ml to 2.3 μg/ml (P=n.s.)). Patients with higher baseline CRP levels were reduced by 58% from 7.2 μg/ml to 3.0 μg/ml (P<0.001). In placebo-treated patients with higher baseline CRP, CRP levels were decreased by 23% (7.1 μg/ml to 5.5 μg/ml).

TABLE II
Low Dose Doxycycline (LDD) Preferentially Suppresses C-Reactive
Protein in Patients With Higher CRP Values at Baseline

| | Patients with lower | | Patients with higher | |
|---|---|---|---|---|
| | baseline CRP (≦5 μg/ml) | | CRP (>5 μg/ml) | |
| | Placebo | LDD | Placebo | LDD |
| Baseline CRP | 2.9 ± 0.6 | 3.0 ± 0.4 | 7.1 ± 0.8 | 7.2 ± 0.6 |
| Six Month CRP | 4.2 ± 0.9^{b} | 2.3 ± 0.5^{b} | 5.5 ± 1.1^{b} | 3.0 ± 0.7^{a} |
| Reduction Due | | 23% | 23% | 58% |
| To Treatment | | | | |

^{a}P < 0.001
^{b}n.s.

1. A method for decreasing C-reactive protein levels in a mammal in need thereof, the method comprising administering to the mammal an effective amount of a non-antibacterial tetracycline formulation.

2. The method according to claim 1, wherein the tetracycline formulation comprises a non-antibacterial amount of an antibacterial tetracycline.

3. The method according to claim 2, wherein the antibacterial tetracycline is selected from the group consisting of terramycin, aureomycin, doxycycline, minocycline, tetracycline, oxytetracycline, chlortetracycline, demeclocycline, lymecycline, or pharmaceutically acceptable salts thereof.

4. The method according to claim 1, wherein the tetracycline formulation comprises a non-antibacterial tetracycline

5. The method of claim 4, wherein the non-antibacterial tetracycline is selected from the group consisting of CMT-1, CMT-2, CMT4, CMT-6, CMT-7 or CMT-9, or pharmaceutically acceptable salts thereof.

6. The method of claim 4, wherein the tetracycline is CMT-3, or its analogs, or pharmaceutically acceptable salts thereof.

7. The method according to claim 4, wherein the tetracycline is CMT-8, or its analogs, or pharmaceutically acceptable salts thereof.

8. The method according to claim 4, wherein the tetracycline is CMT-10, or its analogs, or pharmaceutically acceptable salts thereof.
